(19) United States
(12) Patent Application Publication
(10) Pub. No.: US 2002/0151482 A1
(43) Pub. Date: Oct. 17, 2002

(54) Plasminogen-related gene B Polypeptides

(76) Inventors: Lawrence Weissbach, Boston, MA (US); Valerae Lewis, Houston, TX (US); Michael O'Reilly, Houston, TX (US)

Correspondence Address: JANIS K. FRASER, PH.D., J.D., Fish & Richardson P.C., 225 Franklin Street, Boston, MA, 02110-2804, US

(21) Appl. No.: 09/850,564

(22) Filed: May 7, 2001

Related U.S. Application Data
(63) Continuation of application No. 09/659,277, filed on Sep. 12, 2000, now abandoned.
Application No. 09/659,277 is a continuation of application No. PCT/US99/05155, filed on Mar. 10, 1999.
(60) Provisional application No. 60/077,889, filed on Mar. 13, 1998.

(51) Int. Cl. A61K 48/00; A61K 38/17

(52) U.S. Cl. 514/12; 514/44

(57) ABSTRACT

A method of inhibiting angiogenesis or neoplastic growth, or both, in a bird or mammal is disclosed. The method involves identifying an animal that has, or is at risk for, unwanted angiogenesis or neoplastic growth or both, and administering to the animal an amount of a plasminogen-related gene-B polypeptide sufficient to inhibit the angiogenesis or neoplastic growth.

### CROSS-REFERENCE TO RELATED APPLICATIONS

This application is a continuation of International Application No. PCT,US99/05155, filed Mar. 10. 1999. which claims priority from U.S. Provisional Application No. 60/077.889, filed Mar. 13, 1998, now abandoned.

### STATEMENT AS TO FEDERALLY SPONSORED RESEARCH

This invention was made with Government support under National Institutes of Health grant AM16265. The Government has certain rights in the invention.

### FIELD OF THE INVENTION

The invention relates to cell biology, angiogenesis, and oncology.

### BACKGROUND

Plasminogen, a serine proteinase precursor secreted by the liver, plays a role in dissolving blood clots. The active two-chain proteinase plasmin is produced from plasminogen by cleavage of a specific Arg-Val peptide bond by tissue-type or urokinase-type plasminogen activator, and the autocatalytic removal of an N-terminal preactivation peptide. Plasmin attacks fibrin, a principal component of clots, thereby facilitating clot lysis. In addition to its well-characterized thrombolytic capability, plasmin, along with other proteinases, has been implicated as a mediator of tissue remodeling during development, cartilage destruction in osteoarthritis, and basement membrane invasion by metastatic tumor cells.

A plasminogen-related gene B polypeptide (PRG-B polypeptide; also known as plasmilar or plasminogen-related protein [PRP]) has been described by Weissbach. U.S. Pat. No. 5,545.717; Weissbach et al., Biochem. Biophys. Res. Commun. 186:1108 (1992); and Ichinose et al., Biochemistry 31:3113-3118 (1992). After cleavage of the signal peptide, the mature PRG-B polypeptide is 77 amino acids in length and is highly homologous to the N-terminal preactivation peptide of plasminogen.

### SUMMARY

It has been discovered that a PRG-B polypeptide inhibits angiogenesis in an in vitro avian model, and also inhibits neoplastic growth in an in vivo mammalian model.

Accordingly, the invention features a method of inhibiting angiogenesis in a bird or mammal. e.g., a human. The method involves identifying a bird or mammal that has, or is at risk for, unwanted angiogenesis; and administering to the animal an amount of the PRG-B polypeptide sufficient to inhibit angiogenesis. Conditions involving unwanted angiogenesis include non-cancerous growths, arthritis, and diabetic retinopathy.

The invention also features a method for decreasing or preventing a neoplastic growth such as a cancer of the lung, breast, or prostate gland. The method involves identifying an animal that has, or is at risk for, a neoplastic growth; and administering to the animal an amount of PRG-B polypeptide sufficient to decrease or prevent the neoplastic growth.

The invention further includes (1) a pharmaceutical composition for treating a condition associated with unwanted angiogenesis or neoplastic growth in a subject, the composition including a PRG-B polypeptide; (2) the use of a PRG-B polypeptide for preparation of the pharmaceutical composition: and (3) the use of an expression vector encoding a PRG-B polypeptide for preparation of the pharmaceutical composition.

The PRG-B polypeptide used in the methods of this invention can be full length, mature plasmilar, whose amino acid sequence is disclosed by Weissbach et al., Biochem. Biophys. Res. Commun. 186:1108 (1992) (GenBank Accession No. M93143). Alternatively, the PRG-B polypeptide can be the functional equivalent of plasmilar. i.e., a peptide mimetic based on plasmilar, or a polypeptide that: (1) displays antiangiogenic or anticancer activity, and (2) includes an amino acid sequence sharing at least 70% identity with the full length, mature plasmilar amino acid sequence. Preferably, sequence identity is at least 80%, and more preferably, at least 90%.

A specific example of a PRG-B polypeptide useful in the methods of this invention consists of the following amino acid sequence:

MRGSHHHHHH TDPHASSVPR VDPLDDYVNT QGPSLFSVTK KQLGAGSREE CAAKCEEDKE FTCRAFQYHS KEQQCVIMAE NRKSSIIIRM RDAVLFEK(SEQ ID NO:4).

In this particular sequence (SEQ ID NO:4), the amino-terminal glutamate residue (E) of full-length, mature plasmilar (position 22) is replaced with an aspartate residue (D). In this sequence (SEQ ID NO:4), the six contiguous histidine residues (positions 5-10) function as a “polyhistidine tag” useful in purification of the recombinant polypeptide (by means of its interaction with nickel-NTA agarose). The remaining amino acid residues between the amino terminal methionine of the polypeptide and the amino terminus of the full-length, mature plasmilar are artifacts of DNA cloning procedures used to product the recombinant PRG-B polypeptide.

As used herein. “sequence identity” means the percentage of identical subunits at corresponding positions in two sequences when the two sequences are aligned to maximize subunit matching, i.e., taking into account gaps and insertions. For example, if 7 positions in a sequence 10 amino acids are identical to the corresponding positions in a second 10-amino acid sequence, the two sequences have 70% sequence identity. Sequence identity is typically measured using sequence analysis software such as the Sequence Analysis Software Package of the Genetics Computer Group. University of Wisconsin Biotechnology Center. 1710 University Avenue, Madison, Wis. 53705, or BLAST programs, provided as a service by the National Center for Biotechnology Information, are useful for making sequence comparisons. The programs are described in detail by Karlin et al., Proc Natl Acad Sci USA 87:2264 (1990) and 90:5873 (1993), and Altschul et al., Nucl Acids Res 25:3389 (1997), and are available on the Internet at http://www.ncbi.nlm.nih.gov.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12. and a gap penalty of 4 can be used.

The percent identity between two sequences can be determined using techniques similar to those described above, with or without allowing gaps. In calculating percent identity, only exact matches are counted.

In the case of polypeptide sequences that are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid asparagine and glutamine; serine and threonine: tysine and arginine; and phenylalanine and tyrosine. For example a 10 amino acid polypeptide is said to be at least 80% conserved if it differs from a reference polypeptide by no more than two non-conservative substitutions.

PRG-B polypeptides can be administered directly. Direct administration of PRG-B polypeptides can be systemic, for example, intravenous. Alternatively, PRG-B polypeptides can be administered locally, for example, by direct injection into a tumor. Direct administration also can be from an implant, which provides continuous slow release of the PRG-B polypeptide.

In addition, PRG-B polypeptides can be administered indirectly by means of an expression vector. An expression vector is any nucleic acid molecule or virus containing regulatory elements or reporter genes for the purpose of expression of a given gene in prokaryotic or eukaryotic cells or organisms. Such vectors can be introduced into a cell by means of molecular biological techniques. After introduction into the cell, this nucleic acid can exist extrachromosomally or become integrated into the host genome. Such cells can also be administered to the animal.

One or more additional compounds, including other angiogenesis inhibitors or antimitotic drugs, can be administered before, concurrently, or after administration of a PRG-B polypeptide. When these compounds are administered concurrently, they can be present in one single pharmaceutical composition.

Other features and advantages of the invention will be apparent from the following detailed description, and from the claims.

### BRIEF DESCRIPTION OF DRAWING

FIG. 1 is a graph of mice tumor volume versus days of treatment with saline or PRG-B polypeptide.

### DETAILED DESCRIPTION

The presence of blood vessels in non-cartilaginous tissues of birds or mammals is maintained by a delicate balance of inhibitors and stimulators of angiogenesis. Changes in this balance can promote or inhibit development of blood vessels. The discovery that a PRG-B polypeptide plays a role in the regulation of angiogenesis, and thus tumor growth, has led to the methods of this invention.

Amounts of a PRG-B polypeptide suitable for use in this invention can be produced by conventional recombinant methods employing cultured host cells. For example. recombinant PRG-B polypeptides (rPRG-B) can be produced as described in Example 1 below. The host cells can be prokaryotes. e.g., E. Coli. Alternatively, the host cells can be eukaryotes, e.g., yeast, insect cells, or mammalian cells. Nucleic acid vectors containing a rPRG-B coding region operably linked to suitable expression control sequences can be introduced into the host cells by viral infection, receptor-mediated endocytosis, liposome fusion, or any other standard technique. Extraction and purification of rPRG-B expressed by cultured host cells can be carried out using techniques known in the art, including, for example, the affinity purification procedure described in Example 1 below.

Administration of PRG-B polypeptides according to the invention can be carried out according to various standard methods, including intravenous, subcutaneous, intraarterial, intraperitoneal, transmucosal, oral, and intrapulmonary administration. In addition, an implant that allows slow release can be used to administer the PRG-B polypeptides to the patient. The PRG-B polypeptides can be administered in combination with one or more additional active agents. e.g., a chemotherapeutic drug such as taxol.

PRG-B polypeptides can be administered in dosages comparable to dosages of other therapeutically administered proteins. Typically, dosage levels will be designed to produce a serum concentration of about 1-100 ng/ml when administered systemically. Alternatively, PRG-B polypeptides can be injected directly into a target tissue such as a tumor. This may result in local concentrations of PRG-B polypeptides above 100 ng/ml while systemic serum concentrations remain below 1 ng/ml. Local delivery techniques are further discussed below. Optimal dosage for a given patient depends on factors such as the patient's weight, age, gender, and treatment indication, and can be determined by one of ordinary skill in the art.

The data obtained from cell culture assays and animal studies can be used in formulating a range of PRG-B polypeptide dosage for use in humans. For example, a titration curve can be determined by repeating the procedures described in Example 1 below with varying concentrations of rPRG-B. The dosage lies preferably within a range of circulating concentrations that include the ED₅₀ with little or no toxicity. The dosage may vary within this range depending upon the dosage form employed and the route of administration utilized. Levels of PRG-B polypeptides in plasma can be measured by conventional analytical methods, e.g., high performance liquid chromatography or radioimmunoassay.

Additional guidance on methods of determining dosages can be found in standard references, for example, Spilker, Guide to Clinical Studies and Developing Protocols, Raven Press Books, Ltd., New York. 1984. pp. 7-13 and 54-60; Spilker, Guide to Clinical Trials, Raven Press, Ltd., New York. 1991. pp. 93-101; Craig et al., Modern Pharmacology, 2d ed., Little Brown and Co., Boston. 1986, pp. 127-133; Speight, Avery's Drug Treatment: Principles and Practice of Clinical Pharmacology and Therapeutics, 3d ed., Williams and Wilkins, Baltimore, 1987. pp. 50-56; Tallarida et al., Principles in General Pharmacology, Springer-Verlag, New York. 1998. pp. 18-20. and Olson. Clinical Pharmacology Made Ridiculously Simple, MedMaster, Inc., Miami, 1993. pp. 1-5.

Pharmaceutical compositions containing a PRG-B polypeptide for use in the methods of the present invention can be formulated in conventional manner using one or more physiologically acceptable carriers or excipients.

PRG-B polypeptides may be formulated for administration by inhalation or insufflation (either through the mouth or the nose) or oral, buccal, pulmonary, nasal. parenteral, or rectal administration.

For oral administration, the pharmaceutical compositions may take the form of, for example, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol or fractionated vegetable oils); and preservatives (for example, methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. To avoid degradation of PRG-B polyp eptides in the gastrointestinal tract. delivery systems such as bacterial toxin fusion proteins or liposomes can be used. Such delivery systems are described in Mestecky et al., Behring Inst. Mitt. 98:33-43 (1997); Storm et al., Hybridoma 16:119-125 (1997); and Rowlinson-Busza et al., Curr. Opin. Oncol. 4:1142-1148 (1992).

For buccal administration the compositions may take the form of tablets or lozenges formulated in conventional manner.

For administration by inhalation, the PRG-B polypeptides used according to the methods of the present invention is conveniently delivered in the form of an aerosol spray from pressurized packs or a nebulizer, with the use of a suitable propellant. In the case of a pressurized aerosol the dosage unit may be determined by providing a valve to deliver a metered amount. Capsules and cartridges of, for example, gelatin for use in an inhaler or insufflator may be formulated containing a powder mix of the compound and a suitable powder base such as lactose or starch.

The PRG-B polypeptides can be formulated for parenteral administration by injection, for example, by bolus injection or continuous infusion. Formulations for injection may be presented in unit dosage form, for example, in ampules or in multi-dose containers, with an added preservative. The compositions may take such forms as suspensions. solutions or emulsions in oily or aqueous vehicles, and may contain formulatory agents such as suspending, stabilizing and/or dispersing agents. Alternatively, the active ingredient may be in powder form for constitution with a suitable vehicle, for example, sterile pyrogen-free water, before use.

PRG-B polypeptides can also be formulated in rectal compositions such as suppositories or retention enemas, for example, containing conventional suppository bases such as cocoa butter or other glycerides.

In addition to the formulations described previously, PRG-B polypeptides may also be formulated as a depot preparation (e.g., an implant). Such long acting formulations may be administered by implantation (for example subcutaneously or intramuscularly) or by intramuscular injection. Thus, for example, PRG-B polypeptides can be formulated with suitable polymeric or hydrophobic materials (for example as an emulsion in an acceptable oil) or ion exchange resins, or as sparingly soluble derivatives, for example, as a sparingly soluble salt.

Targeting of a PRG-B polypeptide to neoplastic cells can be achieved by local injection of PRG-B polypeptide compositions directly into tumors. For enhanced targeting, the compositions can contain components that specifically bind to neoplastic cells, e.g., antibodies or polypeptides specific for cancer antigens, preferably cell-surface cancer antigens. Alternatively. PRG-B polypeptides can be targeted to sites of abnormally high angiogenesis by including in the composition polypeptides, e.g., antibodies or fragments thereof, that bind to antigens associated with sites of angiogenesis.

PRG-B polypeptides can also be introduced into a patient by expressing within the cells of the patient a nucleic acid construct containing expression control sequences operably linked to a sequence encoding a PRG-B polypeptide. The nucleic acid construct is derived from a non-replicating linear or circular DNA or RNA vector, or from an autonomously replicating plasmid or viral vector: or the construct is integrated into the host genome. Any vector that can transfect a mammalian cell may be used in the methods of the invention. Methods for constructing expression vectors are well known in the art (see, e.g., Molecular Cloning: A Laboratory Manual, Sambrook et al., eds., Cold Spring Harbor Laboratory, 2nd Edition, Cold Spring Harbor, N.Y. 1989).

In these vectors, promoters are operably linked to the nucleic acid sequence encoding PRG-B polypeptides. Any promoter that can direct a high level of transcription initiation in the target cells may be used in the invention. Such target cells include cancer cells, healthy cells surrounding cancer cells, and any other cell type in close proximity to the area affected. Non-tissue specific promoters, such as the cytomegalovirus (DeBernardi et al., Proc. Natl. Acad. Sci. USA 88:9257-9261, 1991, and references therein), mouse metallothionine I gene (Hammer et al., J. Mol. Appl. Gen. 1:273-288, 1982), HSV thymidine kinase (McKnight, Cell 31:355-365, 1982), and SV40 early (Benoist et al., Nature 290:304-310. 1981) promoters may be used in methods of the invention, as expression of PRG-B polypeptides in the methods of the invention would not be expected to adversely affect transfected cells. The above-described nucleic acid constructs and vectors can be introduced into target cells by any standard method: e.g., as naked DNA, or by liposome fusion, biolistic transfer. electroporation, erythrocyte ghosts, or microsphere methods (microparticles: see. e.g., U.S. Pat. No. 4,789,734; U.S. Pat. No. 4,925,673: U.S. Pat. No. 3,625,214: Gregoriadis, Drug Carriers in Biology and Medicine, pp. 287-341, Academic Press, 1979).

Alternatively, one can employ a viral-based vector as a means for introducing the nucleic acid into the cells of the animal. Preferred viral vectors include those derived from replication-defective hepatitis viruses (e.g., HBV and HCV), retroviruses (see. e.g., WO89/07136; Rosenberg et al., N. Eng. J. Med. 323(9):570-578, 1990). adenovirus (see. e.g., Morsey et al., J. Cell. Biochem., Supp. 17E, 1993), adeno-associated virus (Kotin et al., Proc. Natl. Acad. Sci. USA 87:2211-2215, 1990), replication defective herpes simplex viruses (HSV; Lu et al., Abstract, page 66, Abstracts of the Meeting on Gene Therapy, Sep. 22-26, 1992, Cold Spring Harbor Laboratory, Cold Spring Harbor, N.Y.), canary pox virus. and any modified versions of these vectors.

Rather than introduction of the naked vector into the patient, delivery of PRG-B polypeptides can be accomplished by transfecting cells (e.g., primary cells of the patient) in vitro with an expression vector encoding PRG-B polypeptides: culturing the cells to produce a stably transformed, uniform population; and implanting the PRG-B polypeptide-secreting population of cells into the patient.

Whichever method is used to deliver PRG-B polypeptides into a patient, response to treatment can be measured by various suitable methods known in the art. For example, a solid tumor can be imaged using magnetic resonance imaging techniques after PRG-B polypeptide administration to determine efficacy. Alternatively, a surrogate marker for tumor growth, such as carcino-embryonic antigen or prostate-specific antigen, can be measured in patient samples such as a blood sample. In non-cancerous conditions, angiogenesis can be monitored before and after PRG-B polypeptide administration by direct examination of the affected tissue or using dyes or radioactive tracers to image blood flow. Possible adverse effects of PRG-B polypeptide administration can include inhibition of wound healing, which should be monitored as well.

Contraindications for administration of a PRG-B polypeptide include situations in which inhibiting angiogenesis is especially detrimental to the patient. For example, caution should be exercised when administering a PRG-B polypeptide to infants, pregnant women, or patients experiencing difficulty in wound healing.

One skilled in the art can, based on the above disclosure and the examples described below, utilize the present invention to its fullest extent. The following examples are to be construed as merely illustrative of how one skilled in the art can make and use PRG-B polypeptides and are not limitative of the remainder of the disclosure in any way. Any publications cited in this disclosure are hereby incorporated by reference.

### EXAMPLE 1

### Production of Recombinant PRG-B Polypeptides

A recombinant fusion protein consisting of the PRG-B polypeptide fused to a hexahistidine-containing peptide at the N-terminus was constructed (SEQ ID NO:4). The hexahistidine feature allowed purification of the recombinantly produced polypeptide by means of a nickel-NTA resin (Qiagen).

PCR was performed using the full-length PRG-B cDNA (as described by Weissbach et al.; GenBank Accession No. M93143) as a template and two oligonucleotide primers harboring internal restriction sites, that facilitated subcloning into the pQE-31 plasmid vector (Qiagen). The pQE vectors expressed inserted genes under the control of the E. coli phage T5 promoter and the lac operator. The sense primer was 5′-ACTTCACCCGGGCAAGT CGACCCTCTGGATGAC-3′ (SEQ ID NO:1), corresponding to nucleotides 107-139 of the cDNA: and the antisense primer was 5′-TTCGGATCCCAGTCTAGAACTCTGAAAG-3′ (SEQ ID NO:2), corresponding to nucleotides 365-392 of the cDNA. The PCR was performed using a BIOSCYCLER (IBI) with the following cycle parameters: 94° C. for 90 seconds, 60° C. for 60 seconds, and 72° C. for 30 seconds. A total of 30 cycles were performed. The 286 bp PCR product and the pQE-31 plasmid were digested with BamHI and SalI restriction enzymes, passed over a Chromaspin-100 pre-packed spin filtration column (Clontech) to remove the small DNA fragments released by the enzyme digestions, and ligated together using T4 DNA ligase.

Ligated DNA was transformed into M15 cells containing the pREP4 plasmid (Qiagen), which expresses high levels of the lac repressor, ensuring tight control over transcription of inserted genes. Transformants were analyzed by BamHI/SalI digestion, and insert-containing plasmid clones were sequenced using a primer available from Qiagen (5′-CGGATAACAATTTCACACAG-3′) (SEQ ID NO:3), which lies 27 bp upstream from the initiating methionine codon. One DNA isolate containing the correct sequence in frame with the histidine tag was used for large scale purification of the PRG-B recombinant fusion protein. The DNA sequence predicted a fusion protein of 11,221 daltons and an isoelectric point of 7.54.

Purification of the fusion protein under denaturing conditions was accomplished as follows: Ten milliliters of an overnight culture of the M15 cells harboring the recombinant plasmid were added to 500 ml of LB media containing ampicillin and kanamycin, and shaken at 37° C. until the OD₆₀₀ was 0.8-0.9. IPTG was added to a final concentration of 1 mM, and the culture was shaken for an additional three to four hours.

The cells were harvested by centrifugation, and the cell pellet was dissolved in 5 ml of Buffer A (8 M urea, 0.1 M NaH₂PO₄, 0.01 M imidazole, 10 mM β-mercaptoethanol, and 0.01 M Tris at pH 8.0) per gram wet weight. The solution was stirred for one hour at room temperature, and then centrifuged at 20,000×g for 15 minutes. Three milliliters of a 50% slurry of Ni-NTA resin, previously equilibrated in Buffer A, were added to the supernatant, and the solution was again stirred at room temperature for one hour. The mixture containing the resin and the bacterial extract was loaded into an empty column (14 cm×3.5 cm) and washed sequentially with 50 ml of Buffer A and 10 column volumes of Buffer B (8 M urea, 0.1 M NaH₂PO₄, 0.01 M imidazole. 10 mM β-mercaptoethanol, and 0.01 M Tris at pH 6.25). Material bound to the resin was eluted with six column volumes of Buffer C (8 M urea, 0.1 M NaH₂PO₄, 0.25 M imidazole. 10 mM β-mercaptoethanol, and 0.01 M Tris at pH 6.25). Three milliliter fractions were collected, and ten milliliters of each fraction was analyzed by sodium dodecylsulfate electrophoresis (SDS-PAGE) for the presence of the PRG-B fusion protein. The purified recombinant PRG-B polypeptide (rPRG-B) was dialyzed against PBS before its use in additional examples below.

### EXAMPLE 2

### rPRG-B Inhibits the Growth of Malignant Tumors

Syngeneic C57B16/J mice were implanted with Lewis lung carcinomas as described in O'Reilly et al., Cell 79:315-328 (1994). A suspension of 1×10⁶ tumor cells in 100 microliters of PBS was injected into the subcutaneous dorsa of the mice. Tumors were measured with a dial-caliper, and volumes were determined using the formula: width²×length×0.52. This formula corresponds to the general formula for the volume of an ellipsoid sphere, where 0.52 approximates π/6, and is a standard calculation for estimating tumor volumes.

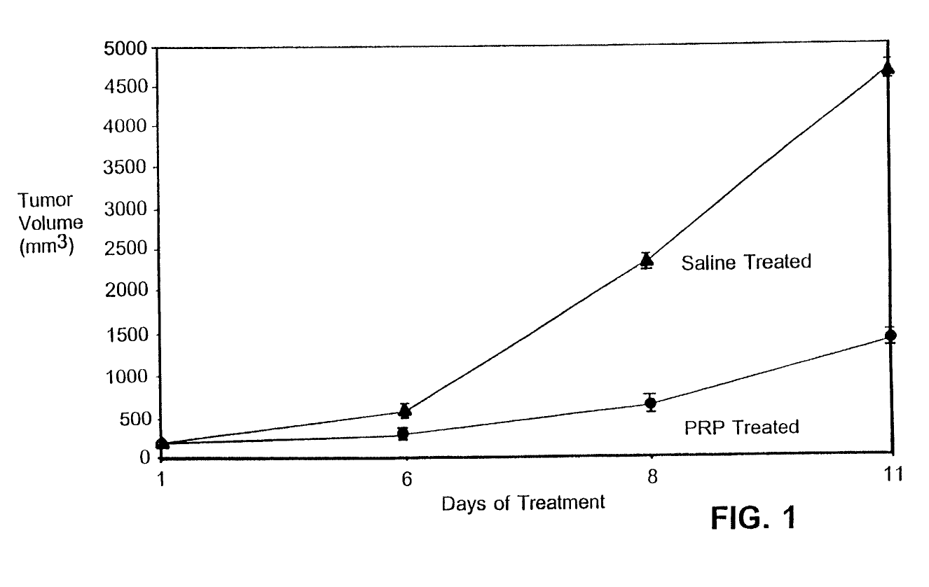

When the tumor volume reached about 160 mm³, the mice were randomized into two groups. One group (n=3) received daily intraperitoneal injections of rPRG-B suspended in PBS (as described in Example 1 above) at the dose of 35 mg/kg body weight. The control group (n=3) received PBS. As shown in FIG. 1, the rPRG-B treatment led to substantial decreases of average tumor size during the observation period. After 11 days of rPRG-B treatment, the average tumor size was less than 1500 mm³. In contrast, the average tumor size was greater than 4500 mm when the mice were treated with saline only. The error bars in the graph represent the 95% confidence interval for each data point. No toxicity or weight loss was observed in any of the treated mice.

### EXAMPLE 3

### rPRG-B Inhibits Angiogenesis

Angiogenesis inhibiting activity of rPRG-B was examined using a standard chick chorioallantoic membrane assay as described in Folkman. Angiogenesis and Its Inhibitors, In: Important Advances in Oncology, De Vila et al. editors, B. Lippincott Co., Philadelphia, pages 42-62 (1985).

Three-day old fertilized white Leghorn eggs (Spafas. Norwich. Conn.) were cracked, and the embryos with intact yolks were placed in petri-dishes. The embryos were incubated for three days at 37° C. and 3% carbon dioxide. After incubation, a methlycellulose disc containing 200 micrograms rPRG-B or nothing was placed onto the chorioallantoic membrane of each embryo. After 48 hours the membranes were examined under a stereomicroscope for the presence or absence of new blood vessels in the tissue under and around the disc. No blood vessels were observed in the tissue under or around the disk at the dosage of 200 micrograms for at least three days after administering rPRG-B. In contrast, the tissue under and round the control discs were heavily infiltrated with new blood vessels. No toxicity was observed in any of the chick embryos.

A number of embodiments of the invention have been described. Nevertheless, it will be understood that various modifications may be made without departing from the spirit and scope of the invention.

What is claimed is:

1. A method of inhibiting angiogenesis in a bird or mammal, the method comprising
identifying a bird or mammal that has, or is at risk for unwanted angiogenesis; and
administering to the bird or mammal an amount of PRG-B polypeptide sufficient to inhibit the unwanted angiogenesis.

2. The method of claim 1, wherein the PRG-B polypeptide is administered to the bird or mammal directly.

3. The method of claim 1, wherein the PRG-B polypeptide is administered indirectly by introducing into the bird or mammal an expression vector encoding a PRG-B polypeptide.

4. The method of claim 1, wherein the PRG-B polypeptide is administered systemically.

5. The method of claim 4, wherein the PRG-B polypeptide is administered intravenously.

6. The method of claim 1, wherein the PRG-B polypeptide is administered locally.

7. The method of claim 1, wherein the PRG-B polypeptide is administered via an implant.

8. The method of claim 1, wherein the mammal is a human.

9. A method of inhibiting neoplastic growth, the method comprising:
identifying a bird or mammal that has or is at risk for neoplastic growth; and
administering to the bird or mammal an amount of PRG-B polypeptide sufficient to inhibit the neoplastic growth.

10. The method of claim 9, wherein the PRG-B polypeptide is administered to the bird or mammal directly.

11. The method of claim 9, wherein the neoplastic growth is selected from the group consisting of lung cancer, breast cancer, and prostate cancer.

12. The method of claim 9, wherein the PRG-B polypeptide is administered by introducing into the animal an expression vector comprising a nucleotide sequence encoding PRG-B polypeptide.

13. The method of claim 9, wherein the PRG-B polypeptide is administered systemically.

14. The method of claim 13, wherein the PRG-B polypeptide is administered intravenously.

15. The method of claim 9 wherein the PRG-B polypeptide is administered via an implant.

16. The method of claim 9 wherein the PRG-B polypeptide is administered locally.

17. The method of claim 9 wherein the animal is a human.

18. The method of claim 1, wherein the unwanted angiogenesis is selected from the group consisting of a non-cancerous growth, arthritis, and diabetic retinopathy.
